(19) United States
(12) Patent Application Publication
(10) Pub. No.: US 2020/0206436 A1
(43) Pub. Date: Jul. 2, 2020

(54) Plastic injection syringe recovery and processing device

(71) Applicant: Guocheng Qian, Guangzhou City (CN)

(72) Inventor: Guocheng Qian, Guangzhou City (CN)

(21) Appl. No.: 16/812,398

(22) Filed: Mar. 9, 2020

(30) Foreign Application Priority Data
Nov. 26, 2019 (CN) 2019111761367

(51) Int. Cl. A61M 5/32

(52) U.S. Cl. CPC A61M 5/3278; A61M 2005/3282; A61M 2005/3279

(57) ABSTRACT

The invention discloses a plastic injection syringe recovery and treatment device, which includes a work box and a work chamber located in the work box. A feed port is provided through the upper end wall of the work box, and the front and back wall of the work chamber are rotated. A first rotating shaft is located on the lower side of the feeding port, a first squeeze barrel is fixed on the first rotating shaft, and the front and rear cavity walls of the working cavity are provided with a frontal shaft located on the right side of the first rotating shaft. There are two rotating shafts, and a second squeeze barrel is fixed on the second rotating shaft. The present invention can recycle the used plastic syringes, and the residual gas and water in the used syringes are firstly squeezed by a squeezing device. Squeeze out and compress the plastic syringe to reduce its volume, and then crush the syringe through the crushing device to heat and melt the crushed syringe. During the heating process, it also sterilizes the metal needle. Function, after heating and melting, the molten plastic liquid is separated from the metal needle, and the classification and recovery are completed.

### CROSS-REFERENCES TO RELATED APPLICATIONS

The present application claims priority from Chinese application No. 2019111771119 filed on Sep. 19, 2019 which is hereby incorporated by reference in its entirety.

### TECHNICAL FIELD

The invention relates to the field of medical waste processing equipment, and in particular to a plastic injection syringe recovery and processing device.

### BACKGROUND OF THE INVENTION

Disposable syringes are disposable syringes made of high-molecular polypropylene materials. Syringes are generally discarded after use, but because the used needles contain bacteria, they will be used by professionals who treat medical waste. As a result of certain dangers, the disposal of disposable syringes is generally directly discarded, which not only wastes plastic resources, but also brings a certain amount of pressure to the environment. Therefore, a plastic injection syringe recycling treatment device is urgently needed In view of the above problems, the present invention has been improved in view of the above disadvantages.

### BRIEF SUMMARY of THE INVENTION

The technical problem to be solved by the present invention is to provide a plastic injection syringe recovery processing device, which overcomes the above-mentioned problems.

The present invention is achieved by the following technical solutions.

A plastic injection syringe recovery and processing device includes a work box and a work chamber located in the work box. An upper end wall of the work box is provided with a feed port therethrough. A first rotating shaft under the feeding port, a first squeeze barrel is fixed on the first rotating shaft, and a second rotating shaft located on the right side of the first rotating shaft is provided on the front and rear walls of the working chamber. A second squeeze barrel is fixed on the second rotating shaft, an assembly box is fixed on the bottom wall of the working cavity, an assembly cavity is set in the assembly box, and a crushing device is set in the assembly cavity. A processing box is fixed on the bottom wall of the assembly chamber, a transmission chamber is provided in the processing box, a transmission chamber is provided in the processing box, and a heating chamber on the right side of the transmission chamber is provided in the processing box. The heating cavity is opened upward, and the heating cavity is in communication with the transmission cavity through a cavity. A push plate is slidably arranged in the cavity, and the bottom of the processing box is fixed with a heating located below the heating cavity. Plate, a one-way valve is fixed on the right end wall of the processing box, and the assembly cavity is fixed on the place. Right tank collection tank, a left end wall of the collecting tank provided with a rectangular fixing hole;

When a plastic syringe enters the working cavity through the feeding port, the first rotating shaft and the second rotating shaft rotate, driving the first squeeze barrel and the second squeeze barrel to rotate, Residual liquid and gas in the syringe are squeezed out, the syringe is compressed, the crushing device is started, and the compressed syringe is cut and crushed to separate the plastic part from the metal part, which is convenient for heating and the broken object falls into the Inside the heating chamber, the heating chamber is closed. The heating plate starts heating to melt the plastic. After the melting is completed, the metal part is retracted. The push plate slides left and right to push the molten plastic in the heating chamber into the collection. Inside the box, complete separation and recovery.

Further, a motor is fixed on the back cavity wall of the working chamber, a motor shaft is dynamically connected to the lower end surface of the motor, a first gear is fixed on the motor shaft, and the first shaft is fixed on the first shaft. A second gear meshed with a first gear, a third gear meshed with the first gear is fixed on the second rotating shaft, and the first gear rotates when the motor shaft rotates, and the first gear rotates The second gear rotates in the opposite direction to the third gear.

Further, the crushing device includes a fixing block fixedly connected to the left cavity wall of the assembly cavity, the fixing block is provided with a first slide groove, and the first slide groove is slidably provided with a rack. A third rotating shaft located on the upper side of the rack is rotatably provided on the rear cavity wall of the assembly cavity, and a fourth gear meshing with the rack is fixed on the third rotating shaft, and the rack and the first The chute is connected by a first spring. A crushing plate is fixed on the right end wall of the rack. The crushing plate is provided with a second chute. The second chute is provided with a sliding rack. The strip is connected to the second chute through a second spring, and a first crushing blade is fixed on the right end surface of the sliding rack. The crushing device further includes a fixing plate fixedly connected to the upper end surface of the collection box. A third chute is provided in the fixing plate, and a sliding rod is slidably disposed in the third chute. The sliding rod is connected to the third chute through a third spring. Two crushing knives, the sliding rod and the sliding rack are connected by a first pull rope, and when the first crushing knife cuts When the second crushing blade retracted, the second crushing blade when cutting, the first crushing blade away.

Further, a fourth chute is provided on the top wall of the processing box, and the fourth chute opens to the right to communicate with the heating chamber. A closed plate is slidably arranged in the fourth chute, and the closed plate is in contact with the closed plate. The fourth chute is connected by a fourth spring, an electromagnet is fixed on the lower end surface of the closing plate, a card slot is arranged in the right top wall of the processing box, and the closing plate slides right into the card slot. Closing the heating cavity.

Further, a fourth rotating shaft located on the left side of the processing box is rotatably provided on the rear cavity wall of the assembly cavity, a first bevel gear is fixed on the fourth rotating shaft, and a left end wall of the processing box is provided with a through hole. A fifth rotation shaft on the right side of the fourth rotation shaft is provided on the back cavity wall of the transmission cavity, a cam is fixed on the fifth rotation shaft, and the fourth rotation shaft and the fifth rotation shaft pass through the first The pulley is drivingly connected, the first pulley passes through the through hole, a push rod is fixed on the left end surface of the push plate, and the push rod extends to the left of the drive cavity and abuts the cam, so The bottom of the processing box is provided with a fifth chute located on the lower side of the transmission cavity. A first slider is slidably arranged in the fifth chute, and the first slider and the fifth chute pass through the fifth The upper end surface of the first slider is fixedly connected to the lower end surface of the push rod through a connecting rod.

Further, a transmission shaft located on the upper side of the sliding rack is rotatably provided on the rear cavity wall of the assembly cavity, and a sector gear is rotatably provided on the transmission shaft, and the sector gear is engaged with the sliding rack. The transmission shaft is connected with the first shaft through a second pulley, and the transmission shaft is connected with the third shaft through a third pulley.

Further, a fixing rod is fixed on the top of the left end surface of the processing box, and a sixth chute is provided in the fixing rod. The sixth chute penetrates to the right and is connected to the fourth chute. A limiting block extending into the sixth chute is fixed and plays a role of limiting. A metal slider is slidably arranged in the sixth chute, and the metal slider and the sixth chute pass through the first Six spring connections. The metal slider is provided with a rotating cavity. The rotating cavity is provided with a sixth rotating shaft. The sixth rotating shaft extends downwardly into the assembly cavity and the sixth rotating shaft. A second bevel gear is fixedly located on the left side of the first bevel gear. When the metal slider slides to the right, the second bevel gear can mesh with the first bevel gear.

Further, a seventh rotation shaft located on the lower side of the fixed block is provided for rotation in the assembly cavity, the seventh rotation shaft and the third rotation shaft are connected through a fourth pulley, and the seventh rotation shaft is fixedly disposed A reel, the reel is connected to the left end wall of the closing plate by a second pull rope, and a third bevel gear located behind the fourth pulley is fixed on the reel.

Further, an eighth rotating shaft located below the seventh rotating shaft is rotatably provided on the top wall of the assembly cavity, and a fourth bevel gear meshing with the third bevel gear is fixed on the top of the eighth rotating shaft. The eighth rotating shaft and the sixth rotating shaft are connected by a fifth pulley, and the fifth pulley is an elastic belt.

The beneficial effects of the present invention: The present invention can recycle used plastic syringes. First, the residual gas and water in the used syringes are squeezed out by an extrusion device, and the plastic syringes are compressed to reduce their use. The volume is then crushed by the crushing device, and the crushed syringe is heated and melted. During the heating process, it also sterilizes the metal needle. After heating and melting, the molten plastic liquid is melted. Separated from the metal needle to complete the sorting recovery.

### BRIEF DESCRIPTION OF THE DRAWINGS

The present invention will be described in detail below with reference to FIGS. 1-4. For convenience of description, the orientation described below is defined as follows: the up-down-left-right-back direction described below is consistent with the up-down-left-right-left direction of the projection relationship of FIG.

FIG. 1 is a schematic diagram of the overall structure of a plastic injection syringe recovery processing device according to the present invention; FIG.

FIG. 2 is a schematic structural view at A-A in FIG. 1; FIG.

FIG. 3 is a schematic structural diagram at B in FIG. 1; FIG.

FIG. 4 is a schematic structural diagram at C in FIG. 1.

### DETAILED DESCRIPTION OF THE INVENTION

The present invention is further described below with reference to the accompanying drawings and embodiments. The modes of the present invention include but are not limited to the following embodiments.

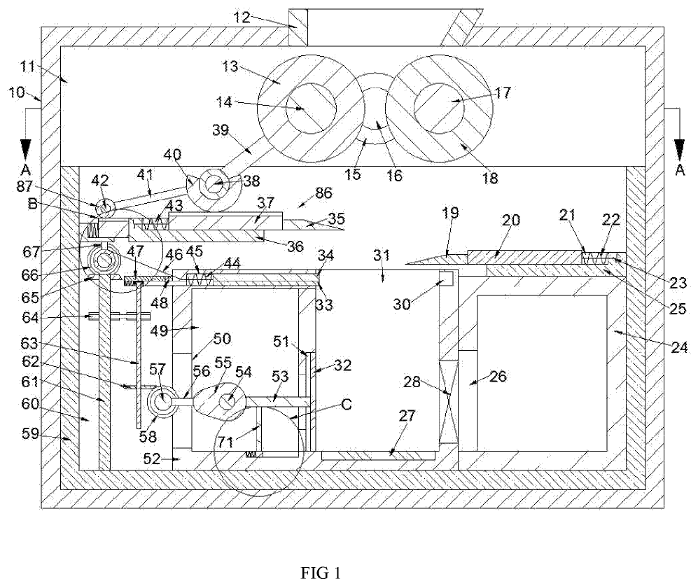

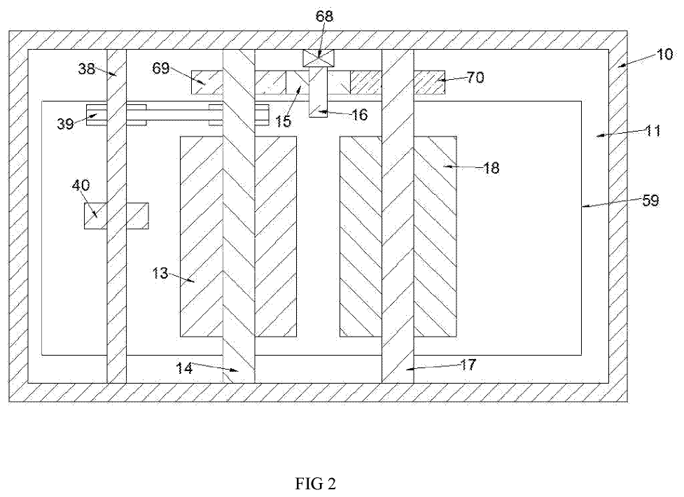

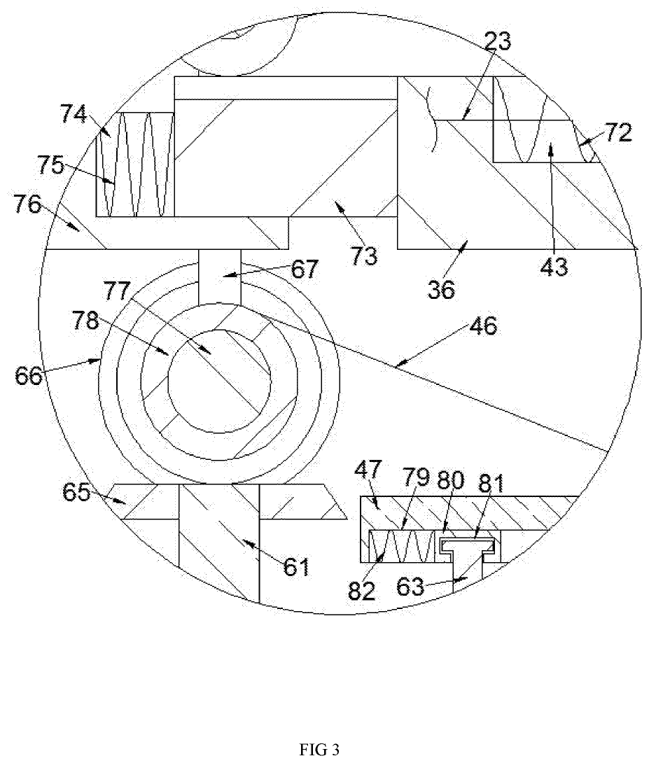

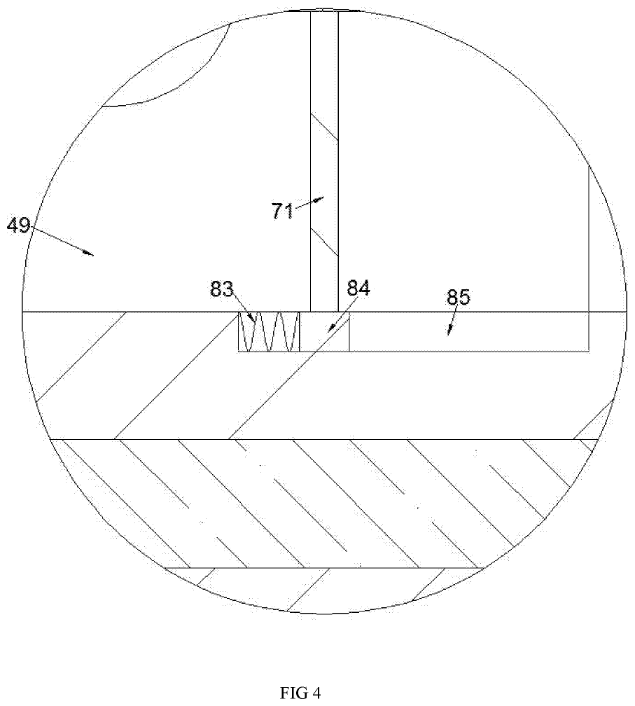

As shown in FIGS. 1-4, a plastic injection syringe recovery and processing device includes a work box 10 and a work chamber 11 located in the work box 10. An upper end wall of the work box 10 is provided with a feed inlet 12, A first rotating shaft 14 located below the feed port 12 is rotatably provided on the front and rear walls of the working cavity 11, and a first squeeze barrel 13 is fixed on the first rotating shaft 14. The front and rear cavities of the working cavity 11 A second rotating shaft 17 is located on the right side of the first rotating shaft 14 and a second squeeze barrel 18 is fixed on the second rotating shaft 17. An assembly box is fixed on the bottom wall of the working chamber 11. 59, the assembly box 59 is provided with an assembly cavity 60 therein, the assembly cavity 60 is provided with a crushing device 86, and a processing box 52 is fixed on the bottom wall of the assembly cavity 60. A transmission cavity 49 is provided, and the processing box 52 is provided with a transmission cavity 49. The processing box 52 is provided with a heating cavity 31 located on the right side of the transmission cavity 49, the heating cavity 31 is opened upward, and the heating The cavity 31 communicates with the transmission cavity 49 through a cavity 51, and a push plate 32 is slidably disposed in the cavity 51. The bottom of the processing box 52 is fixed with a heating located below the heating cavity 31. Plate 27, a one-way valve 28 is fixed on the right end wall of the processing box 52, and a collection box 24 on the right side of the processing box 52 is fixed in the assembly cavity 60, and the left end wall of the collection box 24 is fixed With rectangular holes 26;

When a plastic syringe enters the working chamber 11 through the feeding port 12, the first rotating shaft 14 and the second rotating shaft 17 rotate, driving the first squeeze barrel 13 and the second squeeze shaft. The pressure barrel 18 rotates, squeezes out the remaining liquid and gas in the syringe, compresses the syringe, starts the crushing device 86, cuts and crushes the compressed syringe, separates the plastic part from the metal part, and facilitates heating and crushing. After the object falls into the heating cavity 31, the heating cavity 31 is closed, the heating plate 27 starts heating to melt the plastic, and after the melting is completed, the metal part is stowed, and the push plate 32 slides left and right to move the molten plastic in the heating chamber 31 is pushed into the collection box 24 to complete separation and recovery.

Beneficially, a motor 68 is fixed on the back cavity wall of the working chamber 11, and a motor shaft 16 is dynamically connected to the lower end surface of the motor 68. A first gear 15 is fixed on the motor shaft 16, and the first shaft 14 is fixed. A second gear 69 meshed with the first gear 15 is fixedly mounted thereon, and a third gear 70 meshed with the first gear 15 is fixedly mounted on the second rotation shaft 17. The first gear 15 rotates, and the rotation of the first gear 15 drives the second gear 69 and the third gear 70 to rotate in opposite directions.

Beneficially, the crushing device 86 includes a fixing block 76 fixedly connected to the left cavity wall of the assembly cavity 60. The fixing block 76 is provided with a first sliding groove 74 therein. There is a rack 73, and a third rotating shaft 42 located on the upper side of the rack 73 is rotatably provided on the rear cavity wall of the assembly cavity 60. The third rotating shaft 42 is fixedly provided with a third rotating shaft 42 which is engaged with the rack 73. Four gears 87, the rack 73 and the first slide groove 74 are connected by a first spring 75, a right end wall of the rack 73 is fixed with a crushing plate 36, and a second slide groove is provided in the crushing plate 36 43. A sliding rack 37 is slidably provided in the second sliding groove 43. The sliding rack 37 and the second sliding groove 43 are connected by a second spring 72. The right end surface of the sliding rack 37 is fixedly provided. The first crushing blade 35 and the crushing device 86 further include a fixing plate 25 fixedly connected to the upper end surface of the collection box 24. A third sliding groove 21 is provided in the fixing plate 25. A sliding rod 20 is slidably disposed, and the sliding rod 20 and the third sliding groove 21 are connected by a third spring 22. The left end surface of the sliding rod 20 is fixed with a second crushing blade 19, and the sliding rod 20 is connected to the sliding rack 37 through a first pulling rope 23, when the first crushing knife 35 cuts, the second crushing knife 19 is stowed, and when the second crushing knife 19 is cut, the first crushing knife 35 is stowed.

Beneficially, a fourth chute 45 is provided on the top wall of the processing box 52, and the fourth chute 45 opens to the right to communicate with the heating chamber 31. A closing plate 34 is slidably disposed in the fourth chute 45. The closing plate 34 and the fourth sliding slot 45 are connected by a fourth spring 44. An electromagnet 33 is fixed on the lower end surface of the closing plate 34, and a slot 30 is provided in the right top wall of the processing box 52. The closing plate 34 slides to the right into the slot 30 to close the heating cavity 31.

Beneficially, a fourth rotating shaft 57 located on the left side of the processing box 52 is rotatably provided on the rear cavity wall of the assembly cavity 60, and a first bevel gear 58 is fixed on the fourth rotating shaft 57. The processing box 52 The left end wall is penetratingly provided with a through hole 50. The rear cavity wall of the transmission cavity 49 is rotatably provided with a fifth rotation shaft 54 located on the right side of the fourth rotation shaft 57. The fifth rotation shaft 54 is fixed with a cam 55. The fourth rotating shaft 57 and the fifth rotating shaft 54 are drivingly connected through a first pulley 56, the first pulley 56 passes through the through hole 50, and a push rod 53 is fixed on the left end surface of the push plate 32. The rod 53 extends to the left into the transmission cavity 49, and abuts against the cam 55. The bottom of the processing box 52 is provided with a fifth slide groove 85 located below the transmission cavity 49. The fifth slide groove A first slider 84 is slid in 85, and the first slider 84 and the fifth sliding groove 85 are connected by a fifth spring 83. The upper end surface of the first slider 84 and the lower end surface of the push rod 53 are connected. It is fixedly connected by a link 71.

Beneficially, a transmission shaft 38 located on the upper side of the sliding rack 37 is rotatably provided on the rear cavity wall of the assembly cavity 60, and a sector gear 40 is rotatably disposed on the transmission shaft 38, and the sector gear 40 and the sliding rack 37 meshes, the transmission shaft 38 and the first shaft 14 are drivingly connected by a second pulley 39, and the transmission shaft 38 and the third shaft 42 are drivingly connected by a third pulley 41.

Beneficially, a fixing rod 47 is fixed on the top of the left end surface of the processing box 52, and a sixth sliding slot 79 is provided in the fixing rod 47, and the sixth sliding slot 79 penetrates to the right to the fourth sliding slot 45. Connected, a limiting block 48 extending into the sixth chute 79 is fixed in the fixing rod 47 to play a limiting role. A metal slider 80 is slid in the sixth chute 79. The metal The slider 80 is connected to the sixth chute 79 through a sixth spring 82. The metal slider 80 is provided with a rotation cavity 81 therein. The rotation cavity 81 is provided with a sixth rotation shaft 63 and the sixth rotation shaft. 63 extends downward into the assembly cavity 60, and a second bevel gear 62 located on the left side of the first bevel gear 58 is fixed on the sixth rotating shaft 63. When the metal slider 80 slides to the right At this time, the second bevel gear 62 can mesh with the first bevel gear 58.

Beneficially, a seventh rotation shaft 77 located below the fixing block 76 is provided in the assembly cavity 60 for rotation, and the seventh rotation shaft 77 and the third rotation shaft 42 are connected through a fourth pulley 67. A reel 78 is fixed on the seven rotating shafts 77. The reel 78 and the left end wall of the closing plate 34 are connected by a second rope 46. The reel 78 is fixed on the fourth pulley. 67 [symbol] [symbol] 66. 67 third bevel gear 66.

Beneficially, an eighth rotating shaft 61 located below the seventh rotating shaft 77 is rotatably provided on the top wall of the assembly cavity 60, and a first engaging mesh with the third bevel gear 66 is fixed on the top of the eighth rotating shaft 61. A four-bevel gear 65, the eighth rotating shaft 61 and the sixth rotating shaft 63 are connected by a fifth pulley 64, and the fifth pulley 64 is an elastic belt.

The sequence of mechanical actions of the entire device:

The initial state of the present invention is: the second pulling rope 46 is in a tensioned state, the fourth spring 44 is in a compressed state, the closing plate 34 is located in the fourth chute 45, the electromagnet 33 is not energized, and the second bevel gear 62 and the first The bevel gear 58 is not meshed, the first spring 75 is in a relaxed state, the rack 73 is located in the first sliding groove 74, and the sector gear 40 is not meshed with the sliding rack 37;

1. Put the plastic injection syringe to be recycled into the working chamber 11 through the feed port 12 and start the motor 68;

2. The motor 68 is started to drive the motor shaft 16 to rotate, and the motor shaft 16 is rotated to drive the first gear 15 to rotate. Since the first gear 15 is meshed with the second gear 69 and the third gear 70, the first gear 15 is rotated to drive the second gear 69 and third. The gear 70 rotates, and the second gear 69 and the third gear 70 rotate to drive the first shaft 14 and the second shaft 17 to rotate. The first shaft 14 and the second shaft 17 rotate to drive the first squeeze barrel 13 and the second squeeze barrel 18. Rotate, the first squeeze bucket 13 and the second squeeze bucket 18 rotate to compress the plastic syringe;

3. Because the first rotating shaft 14 and the driving shaft 38 are drivingly connected through the second pulley 39, the first rotating shaft 14 is rotated to drive the driving shaft 38 to rotate, and because the driving shaft 38 and the third rotating shaft 42 are drivingly connected through the third pulley 41, the driving shaft 38 is driven to rotate The third rotation shaft 42 rotates, and the rotation of the third rotation shaft 42 drives the fourth gear 87 to rotate. The fourth gear 87 rotates to move the rack 73 to the right, and the movement of the rack 73 to the right causes the crushing plate 36 to move to the right, and the crushing plate 36 to the right. The movement drives the sliding rack 37 to the right, so that the sliding rack 37 meshes with the sector gear 40;

4. The driving shaft 38 rotates to drive the sector gear 40. The sector gear 40 rotates to cause the sliding rack 37 to slide left and right continuously in the second chute 43, and the sliding rack 37 continuously slides left and right to drive the first crushing knife 35 to slide left and right. 37 is connected to the sliding rod 20 through the first pull rope 23. The sliding rack 37 continuously slides left and right to drive the sliding rod 20 to slide left and right. The sliding rod 20 continues to slide left and right to drive the second crushing knife 19 to slide left and right. Tube for cutting;

5. When the heating chamber 31 is full, the motor 68 starts to reverse, so that the rack 73 moves to the left, and the rack 73 moves to the left to disengage the sector gear 40 from the sliding rack 37;

6. The second pull rope 46 relaxes and resets the fourth spring 44. The return of the fourth spring 44 moves the closing plate 34 to the right, the closing plate 34 moves to the right into the slot 30, closes the heating cavity 31, and the heating plate 27 starts heating, Melting the plastic in the heating cavity 31;

7. After the heating is completed, the electromagnet 33 is energized, and the metal needle in the heating chamber 31 is sucked up. The electromagnet 33 is energized to attract the metal slider 80, which slides the metal slider 80 to the right, and the metal slider 80 slides to the right to drive the sixth rotation shaft. 63 slides to the right, and the sixth rotation shaft 63 slides to the right until the second bevel gear 62 meshes with the first bevel gear 58;

8. Because the third rotating shaft 42 and the seventh rotating shaft 77 are drivingly connected through the fourth pulley 67, the third rotating shaft 42 rotates to drive the seventh rotating shaft 77 to rotate, and the seventh rotating shaft 77 rotates to drive the third bevel gear 66 to rotate. The fourth bevel gear 65 meshes, the third bevel gear 66 rotates to drive the fourth bevel gear 65, and the fourth bevel gear 65 rotates to rotate the eighth rotating shaft 61. Because the eighth rotating shaft 61 and the sixth rotating shaft 63 are transmitted through the fifth pulley 64 Connected, the rotation of the eighth rotation shaft 61 drives the sixth rotation shaft 63 to rotate, and the rotation of the sixth rotation shaft 63 drives the second bevel gear 62 to rotate;

9. Because the second bevel gear 62 and the first bevel gear 58 are in meshing state, the rotation of the second bevel gear 62 drives the first bevel gear 58 to rotate, and the rotation of the first bevel gear 58 drives the fourth shaft 57 to rotate. The five rotation shafts 54 are connected through the transmission of the first pulley 56. The rotation of the fourth rotation shaft 57 drives the rotation of the fifth rotation shaft 54. The rotation of the fifth rotation shaft 54 drives the rotation of the cam 55. The rotation of the cam 55 causes the push rod 53 to move left and right continuously, and the push rod 53 moves continuously left and right The push plate 32 is continuously moved left and right to push the molten plastic in the heating chamber 31 into the collection box 24;

10. When all the plastic cups in the heating chamber 31 are pushed into the collection box 24, the electromagnet 33 is powered off, and the metal needles adsorbed on the electromagnet 33 are transferred into the heating chamber 31 to complete the classification recovery.

The above embodiments are only for explaining the technical concept and characteristics of the present invention, and the purpose thereof is to enable those skilled in the art to understand and implement the content of the present invention, but not to limit the protection scope of the present invention. Any equivalent change or modification made according to the spirit and essence of the present invention should be covered by the protection scope of the present invention.

1. A plastic injection syringe recovery and processing device includes a work box and a work chamber located in the work box. A feed port is formed through an upper end wall of the work box, and is characterized in that: A first rotating shaft is located on the lower side of the feeding port, a first squeeze barrel is fixed on the first rotating shaft, and the front and rear cavity walls of the working cavity are provided with a frontal shaft located on the right side of the first rotating shaft. Two rotating shafts, a second squeeze barrel is fixed on the second rotating shaft, an assembly box is fixed on the bottom wall of the working chamber, an assembly chamber is provided in the assembly box, and an assembly chamber is provided in the assembly chamber. There is a crushing device. A processing box is fixed on the bottom wall of the assembly cavity. The processing box is provided with a transmission cavity. The processing box is provided with a transmission cavity. The processing box is provided with a right side of the transmission cavity. The heating chamber on the side is opened upward, and the heating chamber communicates with the transmission chamber through a cavity. A push plate is slidably arranged in the cavity, and the bottom of the processing box is fixedly located in the heating cavity. A heating plate on the lower side, a one-way valve is fixed on the right end wall of the processing box, and the assembly cavity is fixed inside. The collection bin located on the right side box process, a left end wall of the collecting tank provided with a rectangular fixing hole;
When a plastic syringe enters the working cavity through the feeding port, the first rotating shaft and the second rotating shaft rotate, driving the first squeeze barrel and the second squeeze barrel to rotate, Residual liquid and gas in the syringe are squeezed out, the syringe is compressed, the crushing device is started, and the compressed syringe is cut and crushed to separate the plastic part from the metal part, which is convenient for heating and the broken object falls into the Inside the heating chamber, the heating chamber is closed. The heating plate starts heating to melt the plastic. After the melting is completed, the metal part is retracted. The push plate slides left and right to push the molten plastic in the heating chamber into the collection. Inside the box, complete separation and recovery.

2. The recycling device for plastic injection syringe according to claim 1, characterized in that: a motor is fixed on the back cavity wall of the working chamber, a motor shaft is connected to the lower end of the motor, and the motor shaft is fixed on A first gear is provided, a second gear meshing with the first gear is fixed on the first rotating shaft, and a third gear meshing with the first gear is fixed on the second rotating shaft. The rotation of the motor shaft drives the first gear to rotate, and the rotation of the first gear drives the second gear to rotate in the opposite direction to the third gear.

3. The plastic injection syringe recovery processing device according to claim 1, wherein the crushing device comprises a fixing block fixedly connected to the left cavity wall of the assembly cavity, and a first block is provided in the fixing block. A chute, in which a rack is slidably disposed in the first chute, a third rotating shaft located on the upper side of the rack is rotatably provided on the rear cavity wall of the assembly cavity, and the third rotating shaft is fixedly connected with the third rotating shaft. The fourth gear meshed by the rack, the rack and the first chute are connected by a first spring, a crushing plate is fixed on the right end wall of the rack, and a second chute is provided in the crushing plate, A sliding rack is slidably disposed in the second chute, the sliding rack and the second chute are connected by a second spring, and a first crushing blade is fixed on the right end surface of the sliding rack, and the crushing The device further comprises a fixing plate fixedly connected to the upper end surface of the collection box, a third sliding groove is provided in the fixing plate, and a sliding rod is slidably arranged in the third sliding groove, and the sliding rod and the third The chute is connected by a third spring, and a second crushing knife is fixed on the left end surface of the sliding rod. It is connected to the sliding rack through a first pull rope. When the first crushing knife cuts, the second crushing knife is stowed, and when the second crushing knife is cut, the first crushing knife is stowed.

4. The recycling treatment device for a plastic injection syringe according to claim 1, characterized in that: a fourth chute is provided on the top wall of the processing box, and the fourth chute opens to the right and communicates with the heating chamber, A closing plate is slidably arranged in the fourth chute, and the closing plate and the fourth chute are connected by a fourth spring. An electromagnet is fixed on the lower end surface of the closing plate. A card slot is provided, and the closing plate slides right into the card slot to close the heating cavity.

5. The recycling and processing device for a plastic injection syringe according to claim 1, characterized in that: a fourth rotation shaft located on the left side of the processing box is provided on the rear cavity wall of the assembly cavity, and the fourth rotation shaft is provided on the fourth rotation shaft A first bevel gear is fixedly arranged, a through hole is formed through the left end wall of the processing box, and a fifth shaft located on the right side of the fourth shaft is rotatably provided on the back cavity wall of the transmission cavity. A cam is provided, and the fourth rotating shaft and the fifth rotating shaft are drivingly connected through a first pulley, the first pulley passes through the through hole, and a push rod is fixed on the left end surface of the push plate, and the push rod Extending to the left into the transmission cavity and abutting against the cam, the bottom of the processing box is provided with a fifth chute on the lower side of the transmission cavity, and the first chute is slidably provided in the fifth chute. Block, the first slide block and the fifth slide slot are connected by a fifth spring, and the upper end surface of the first slide block and the lower end surface of the push rod are fixedly connected by a connecting rod.

6. The recycling and processing device for a plastic injection syringe according to claim 3, characterized in that: the rear cavity wall of the assembly cavity is rotatably provided with a transmission shaft located on the upper side of the sliding rack, and the transmission shaft rotates A sector gear is provided, the sector gear is meshed with the sliding rack, the transmission shaft is connected with the first shaft through a second pulley, and the transmission shaft is connected with the third shaft through a third pulley.

7. The plastic injection syringe recovery processing device according to claim 5, characterized in that: a fixed rod is fixed on the top of the left end surface of the processing box, and a sixth chute is provided in the fixed rod, and the sixth The chute penetrates to the right and is connected to the fourth chute. A limiting block extending into the sixth chute is fixed inside the fixed rod, and plays a limiting role. A metal slider, which is connected to the sixth chute through a sixth spring. The metal slider is provided with a rotation cavity. A rotation shaft is provided in the rotation cavity, and the sixth rotation is provided. The axial direction extends downward into the assembly cavity, and a second bevel gear located on the left side of the first bevel gear is fixed on the sixth rotating shaft. When the metal slider slides to the right, the first The two-bevel gear is meshable with the first bevel gear.

8. The recycling and processing device for a plastic injection syringe according to claim 4, characterized in that: a seventh rotary shaft located below the fixed block is rotatably provided in the assembly cavity, and the seventh rotary shaft and the third rotating shaft is connected by a fourth pulley, and a reel is fixed on the seventh rotating shaft. The reel is connected to the left end wall of the closing plate by a second rope. The reel is fixed on the reel. A third bevel gear on the rear side of the fourth pulley.

9. The plastic injection syringe recovery processing device according to claim 7, characterized in that: the top wall of the assembly cavity is rotatably provided with an eighth rotating shaft located below the seventh rotating shaft, and the top of the eighth rotating shaft A fourth bevel gear meshed with the third bevel gear is fixed, the eighth rotating shaft and the sixth rotating shaft are connected through a fifth pulley, and the fifth pulley is an elastic belt.
